(19) United States
(12) Patent Application Publication
(10) Pub. No.: US 2024/0099577 A1
(43) Pub. Date: Mar. 28, 2024

(54) SYSTEM FOR IMAGING AND DIAGNOSIS OF RETINAL DISEASES

(71) Applicant: Alcon Inc., Fribourg (CH)

(72) Inventors: John Park, Irvine, CA (US); Qing Xiang, Irvine, CA (US); Lu Yin, Keller, TX (US); Vignesh Suresh, Arlington, TX (US)

(21) Appl. No.: 18/475,369

(22) Filed: Sep. 27, 2023

Related U.S. Application Data
(60) Provisional application No. 63/377,291, filed on Sep. 27, 2022.

(51) Int. Cl. A61B 3/10; A61B 3/12; G06T 7/00

(52) U.S. Cl. CPC A61B 3/102; A61B 3/12; G06T 7/00; G06T 2207/10036; G06T 2207/20081; G06T 2207/30041

(57) ABSTRACT

In certain embodiments, a system, a computer-implemented method, and computer-readable medium are disclosed for performing integrated analysis of MSI and OCT images to diagnose eye disorders. MSI and OCT are processed using separate input machine learning models to create input feature maps that are input to an intermediate machine learning model. The intermediate machine learning model processes the input feature maps and outputs a final feature map that is processed by one or more output machine learning models that output one or more estimated representations of a pathology of the eye of the patient. A single device captures OCT and non-OCT images using (a) a sensor of a first imaging device that is shared with a second imaging device and/or (b) an optical component for directing light form the retina to the first imaging device or the second imaging device.

### BACKGROUND

Multispectral imaging (MSI) is a technique that involves measuring (or capturing) light from samples (e.g., eye tissues/structures) at different wavelengths or spectral bands across the electromagnetic spectrum. MSI may capture more information from the samples that may not be visible through conventional imaging, which generally uses broadband illumination and a broadband imaging sensor. The MSI information obtained by an MSI imaging system may be used to diagnose eye disorders and to enable real-time adjustment in the use of instruments (e.g., forceps, lasers, probes, etc.) used to manipulate eye tissues/structures during surgery.

Optical coherence tomography (OCT) is a technique that uses light waves to generate two dimensional (2D) and three-dimensional (3D) images of the eye. 2D OCT may involve the use of time-domain OCT and/or Fourier-domain OCT, the latter involving the use of spectral-domain OCT and swept-source OCT methods. 3D OCT may similarly utilize time-domain OCT and Fourier-domain OCT imaging techniques. OCT imaging may likewise be used pre-operatively to diagnose eye disorders or intra-operatively.

It would be an advancement in the art to better utilize the capabilities of MSI and OCT to diagnose eye disorders.

### SUMMARY

In certain embodiments, a system is provided. The system includes a first imaging device configured to capture a first image of a retina of a patient, the first image being an optical coherence tomography (OCT) image. The system further includes a second imaging device configured to capture a second image of the retina of the patient according to an imaging modality other than OCT. In the system, at least one of (a) a sensor of the first imaging device is shared with the second imaging device and (b) an optical component is configured to select which of the first imaging device and the second imaging device receives light reflected from the retina to the first imaging device or the second imaging device.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an example system for performing integrated analysis of MSI and OCT images to diagnose eye disorders in accordance with certain embodiments.

FIG. 2A is diagram illustrating a first approach for training machine learning models to perform integrated analysis of MSI and OCT images to diagnose eye disorders in accordance to certain embodiments.

FIG. 2B is diagram illustrating a second approach for training machine learning models to perform integrated analysis of MSI and OCT images to diagnose eye disorders in accordance to certain embodiments.

FIG. 2C is diagram illustrating a third approach for training machine learning models to perform integrated analysis of MSI and OCT images to diagnose eye disorders in accordance to certain embodiments.

FIG. 3 is a flow diagram of a method for training machine learning models to perform integrated analysis of MSI and OCT images to diagnose eye disorders in accordance to certain embodiments.

FIG. 4A illustrates a system for capturing both OCT and MSI images as well as spectral information in accordance with certain embodiments.

FIG. 4B illustrates an alternative system for capturing both OCT and MSI images as well as spectral information in accordance with certain embodiments.

FIG. 4C is a more detailed diagram of a system for capturing both OCT and MSI images as well as spectral information in accordance with certain embodiments.

FIGS. 5A and 5B are diagrams illustrating systems for diagnosing eye disorders using machine learning models in accordance with certain embodiments.

FIG. 6 illustrates an example computing device that implements, at least partly, one or more functionalities for performing integrated analysis of images of multiple imaging modalities in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Various embodiments described herein provide a framework for processing the information obtained from MSI and OCT images using artificial intelligence. An advantage of MSI is that MSI images contain rich information about the retina within the wide range of spectral bands and these are features that cannot be seen using human vision or a Fundus camera. The wide range of spectral bands of MSI further provides a high degree of depth penetration into the retina. However, an MSI image does not provide structural information. In contrast, OCT images do provide structural information about the retina. However, a high degree of expertise is required to interpret OCT images. Using the approach described herein, the rich detail and high depth penetration of MSI can be combined with the structural information of OCT to identify biomarkers for various pathologies and perform early disease diagnosis.

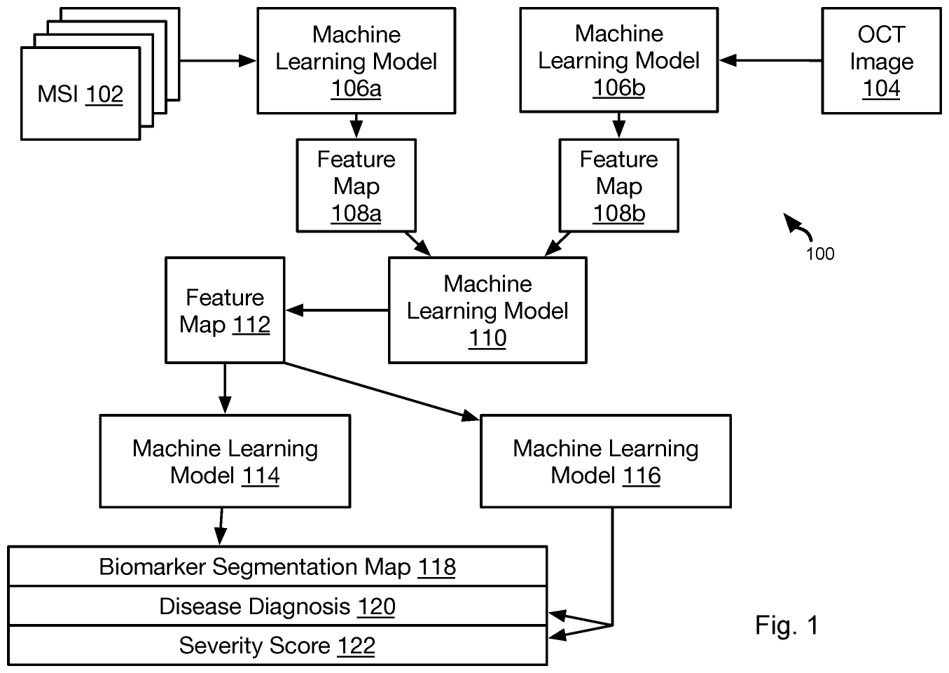

FIG. 1 illustrates a system 100 for performing integrated analysis of MSI images 102 and an OCT image 104. The system 100 may include three main stages, a feature extraction stage using machine learning models 106a, 106b, a feature boosting stage using machine learning model 110, and a biomarker and prediction stage using machine learning models 114, 116. Through those three stages, the system 100 processes MSI images 102 and OCT images 104 separately for feature extraction and then combines the extracted features to obtain meaningful interpretations.

The MSI images 102 may be captured using any approach for implementing MSI known in the art, including so-called hyper-spectral imaging (HSI). Likewise, the OCT image 104 may be obtained using any approach for performing OCT known in the art.

The MSI images 102 are obtained by illuminating the eye of a patient using multi-spectral band illumination sources (e.g., narrowband illumination sources, narrowband filters, etc.) and/or measuring reflected light using multi-spectral band cameras (e.g., an imaging sensor capable of sensing multiple spectral bands, beyond RGB spectral bands). Accordingly, each MSI image 102 represents reflected light within a specific spectral ban. Differences among the MSI images 102 result from different reflectivities of different structures within the eye for different spectral bands. The MSI images 102, when considered collectively, therefore provide additional information about the structures of the eye than a single broadband image. In some implementations, the MSI images 102 are en face images of the retina that are used to detect pathologies of the retina. However, MSI images 102 of other parts of the eye, such as the vitreous or anterior chamber may also be used.

Optical coherence tomography (OCT) is a technique that uses light waves from a coherent light source, i.e., laser, to generate two-dimensional (2D) and three-dimensional (3D) images of the eye. OCT images are typically cross-sectional images of the eye for planes parallel to and colinear with the optical axis of the eye. However, OCT images for a plurality of section planes may be used to construct a 3D image, from which 2D images may be generated for section planes that are not parallel to the optical axis. For example, an en face image of the retina may be derived from the 3D image. In some embodiments, the OCT image 104 is such an en face image of the retina. OCT is capable of imaging the retina up to a certain depth such that the OCT image 104, in some embodiments, is a collection of en face images for image planes at or above the surface of the retina down to a depth within or below the retina.

Although the examples described herein relate to the use of MSI images 102 and OCT images 104, images from any pair of imaging modalities, or images from three or more different imaging modalities, may be used in a like manner. For example, additional imaging modalities may include scanning laser ophthalmology (SLO), a fundus camera, and/or a broadband visible light camera.

In the system 100, the MSI images 102 are processed by a machine learning model 106a and the OCT image 104 is processed by a machine learning model 106b. The machine learning models 106a, 106b may be implemented as a neural network, deep neural network (DNN), convolution neural network (CNN), recurrent neural network (RNN), region-based CNN (R-CNN), autoencoder (AE) or other type of neural network.

The result of processing the images 102, 104 by the machine learning models 106a, 106b are feature maps 108a, 108b, respectively. For example, the feature maps 108a, 108b may be the outputs of one or more hidden layers of the machine learning models 106a, 106b. The feature maps 108a, 108b may be two-dimensional or three-dimensional arrays of values. Where the feature maps 108a, 108b are two-dimensional arrays, the feature maps 108a, 108b may include identical sizes in both dimensions or may be different. Where one or both of the feature maps 108a, 108b is a three-dimensional array, the feature maps 108a, 108b may include identical sizes in at least two dimensions or may be different in any of the three dimensions.

The feature maps 108a, 108b, and possibly the images 102, 104, are processed by a machine learning model 110. The machine learning model 110 may be implemented as a neural network, deep neural network (DNN), convolution neural network (CNN), recurrent neural network (RNN), region-based CNN (R-CNN), autoencoder (AE) or other type of neural network. The result of processing the feature maps 108a, 108b, and possibly the images 102, 104, by the machine learning model 110 is a feature map 112. For example, the feature map 112 may be the outputs of one or more hidden layers of the machine learning model 110 as discussed in greater detail below.

The feature map 112, and possibly the images 102, 104, are then processed by a machine learning model 114 and a machine learning model 116, which then outputs one or more biometric segmentation maps 118, which label features of the eye represented in the images 102, 104 corresponding to one or more pathologies. Each biometric segmentation map 118 may be in the form of an image having the same size as the images 102, 104 and in which non-zero pixels correspond to pixels in the images 102, 104 identified as corresponding to a particular pathology represented by the biometric segmentation map. The biometric segmentation maps 118 may include a separate map for each pathology of a plurality of pathologies or a single map in which all pixels representing any of the plurality of pathologies are non-zero.

The machine learning model 114 may be implemented as a neural network, deep neural network (DNN), convolution neural network (CNN), recurrent neural network (RNN), region-based CNN (R-CNN), autoencoder (AE) or other type of neural network. For example, the machine learning model 114 may be implemented as a U-net.

The machine learning model 116 outputs a disease diagnosis 120 and possibly a severity score 122 corresponding to the disease diagnosis. The machine learning model 116 may be implemented as a long short term memory (LSTM) machine learning model, generative adversarial network (GAN) machine learning model, or other type of machine learning model. The disease diagnosis 120 may be output in the form of text naming the pathology, a numerical code corresponding to the pathology, or some other representation. The severity score 122 may be a numerical value, such as a value from 1 to 10 or a value in some other range. The severity score 122 may be limited to a discrete set of values (e.g., integers from 1 to 10) or may be any value within the limits of precision for the number of bits used to represent the severity score 122.

Pathologies for which biometric segmentation maps 118 may be generated and for which a diagnosis 120 and severity score 122 may be generated include at least those which cause perceptible changes to the retina, such as at least the following:
Retinal tears
Retinal detachment
Diabetic retinopathy
Hypertensive retinopathy
Sickle cell retinopathy
Central retinal vein occlusion
Epiretinal membrane
Macular holes
Macular degeneration (including age-related Macular Degeneration)
Retinal pigmentosa
Glaucoma
Alzheimer's disease
Parkinson's disease

The biometric segmentation maps 118 may, for example, mark vascular features that corresponding to a pathology. Examples of vascular features that can be used to diagnose a pathology are described in the following references, both of which are incorporated herein by reference in their entirety:
Segmenting Retinal Vessels Using a Shallow Segmentation Network to Aid Ophthalmic Analysis, M. Arsalan et al., Mathematics 2022, Volume 10, p. 1536.
PVBM: A Python Vasculature Biomarker Toolbox Based on Retinal Blood Vessel Segmentation, J. Fhima et al., Cornell University (31 Jul. 2022).

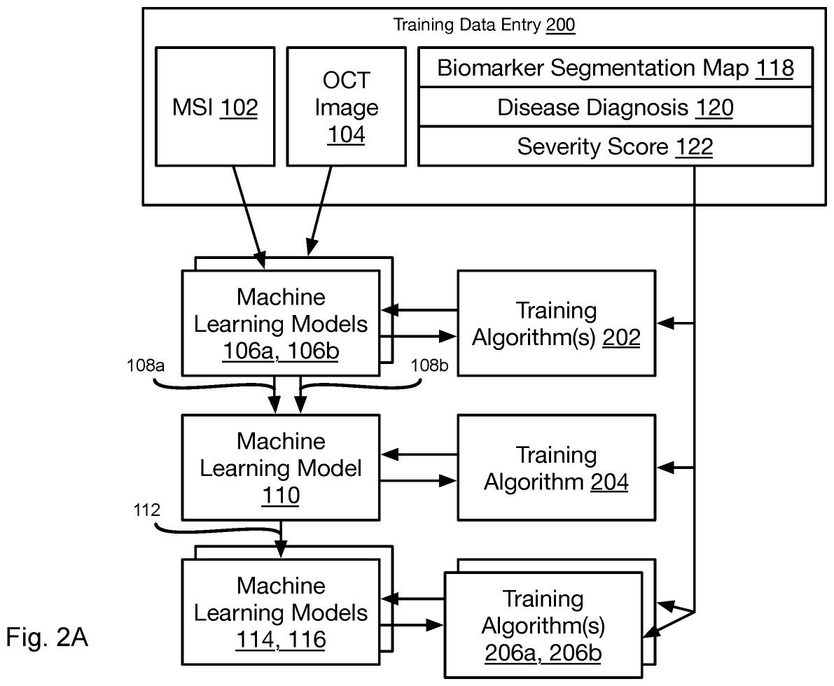

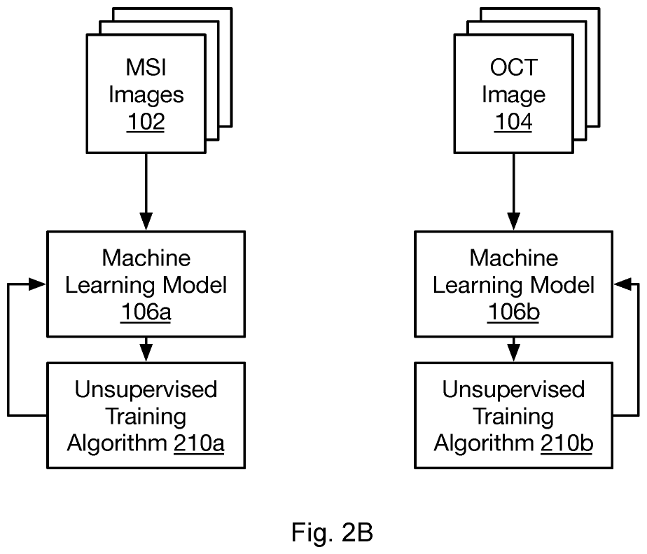

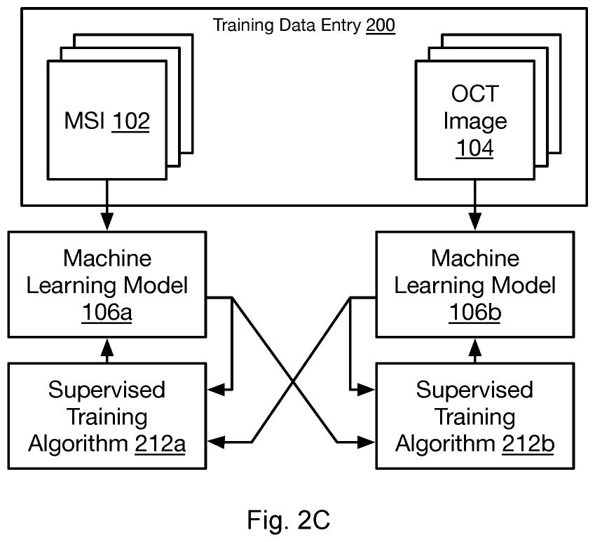

FIG. 2A illustrates an example approach for training the machine learning models 106a, 106b, 110, 114, 116. In particular, FIG. 2A illustrates a supervised machine learning approach that uses a plurality of training data entries 200, such as many hundreds, thousands, tens of thousands, hundreds of thousands, or more. Each training data entry 200 may include, as inputs, MSI images 102 and an OCT image 104. Each image of the MSI images 102 represents an image obtained by detecting light in a different spectral band relative to the other MSI images 102.

The MSI images 102 and OCT image 104 images of a training data entry 200 may be of the same eye of a patient and may be captured substantially simultaneously such that the anatomy represented in the images 102, 104 is substantially the same. For example, “substantially simultaneously” may mean within 1 second and 1 hour of one another. However, “substantially simultaneously” may depend on the pathologies being detected: those that have a very slow progression may use images 102, 104 with longer differences in times of capture, such as less than one day, less than a week, or some other time difference. The MSI images 102 and OCT image 104 are preferably aligned and scaled relative to one another such that a given pixel coordinate in the MSI images 102 represents substantially the same location (e.g., within 0.1 mm, within 1 μm, or within 0.01 μm) in the eye as the same pixel coordinate in the OCT image 104. This alignment and scaling may be achieved for the entire images 102, 104 or for at least a portion of one or both of the images 102, 104 showing anatomy of interest (e.g., the macula of the retina).

Alignment and scaling of the images 102, 104 relative to one another may be achieved by alignment of optical axes of instruments used to capture the images 102, 104 and calibrating the magnification of the instruments to achieve substantially identical scaling (e.g., within +/−0.1%, within 0.01%, or within 0.001%). Alternatively, alignment and scaling of the images 102, 104 may be achieved by analyzing anatomy represented in the images 102, 104. For example, where the MSI images 102 and OCT image 104 represent the retina of the eye, the pattern of blood vessels represented in each image 102, 104 may be used to align and scale one or both of the images 102, 104. Where the images 102, 104 are different sizes or do not completely overlap, such as after registering and scaling, non-overlapping portions of one or both of the images 102, 104 may be trimmed and/or one or both of the images 102, 104 may be padded such that the images 102, 104 are the same size and completely overlap one another.

Each training data entry 200 may include, as desired outputs, some or all of one or more biomarker segmentation maps 118, a disease diagnosis 120, and a severity score 122. A same patient may have multiple pathologies present such that a segmentation map 118, a disease diagnosis 120, and a severity score 122 may be included for each pathology present or a subset of most dominant pathologies. The desired outputs are generated by a human expert based on evaluations of the images 102, 104 and possibly other health information for the patient obtained before or after capture of the images 102, 104. In particular, the biomarker segmentation map 118 for a pathology may include pixels of one or both of the images 102, 104 marked by a human expert as corresponding to the pathology.

For each training data entry 200, the machine learning model 106a receives the MSI images 102 to produce one or more estimated biomarker segmentation maps. For example, the output of the machine learning model 106a may be a three-dimensional array in which each two-dimensional array along a third dimension is an estimated biometric segmentation map corresponding to a pathology.

A training algorithm 202 compares the one or more estimated biomarker segmentation maps to the one or more biomarker segmentation maps 118 for the training data entry 200. The training algorithm 202 then updates one or more parameters of the machine learning model 106a according to differences between each estimated biomarker segmentation map for a pathology and the corresponding biomarker segmentation map 118 for that pathology in the training data entry 200.

The machine learning model 106b may be trained in a like manner to the machine learning model 106a. For each training data entry 200, the machine learning model 106b receives the OCT image 104 and produces one or more estimated biomarker segmentation maps. For example, the output of the machine learning model 106a may be a three-dimensional array in which each two-dimensional array along a third dimension is an estimated biometric segmentation map corresponding to a pathology.

A training algorithm 202, which may be the same as or different from that used to train the machine learning model 106a, compares the one or more estimated biomarker segmentation maps to the one or more biomarker segmentation maps 118 of the training data entry 200. The training algorithm 202 then updates one or more parameters of the machine learning model 106b according to differences between each estimated biomarker segmentation map for a pathology and the corresponding biomarker segmentation map 118 for that pathology in the training data entry 200.

Where three or more imaging modalities are used, additional machine learning models may be present and trained in a like manner. Each machine learning model corresponds to an imaging modality and processes a corresponding image for that imaging modality in the training data entry. The machine learning model produces one or more estimated biomarker segmentation maps that are compared to the one or more biomarker segmentation maps 118 of the training data entry by a training algorithm, which then updates the machine learning model according to the comparison. A hidden layer for each machine learning model may produce outputs that are used as a feature map for the imaging modality to which the machine learning model corresponds.

As described above with respect to FIG. 1, the machine learning model 110 takes as inputs the feature maps 108a, 108b of the machine learning models 106a, 106b. The machine learning model 110 may be trained after the machine learning models 106a, 106b are trained with some or all of the training data entries 200.

For each training data entry 200, the machine learning model 110 receives feature maps 108a, 108b obtained from processing the MSI images 102 and OCT image 104 of the training data entry 200 with the machine learning models 106a, 106b. As noted above, the feature maps 108a, 108b may be the outputs of hidden layers of the machine learning models 106a, 106b, respectively, i.e., a layer other than the final layer that outputs the one or more estimated biomarker segmentation maps. The machine learning model 110 may also receive the MSI images 102 and OCT image 104 as inputs, though in other embodiments, only the feature maps 108a, 108b are used.

The machine learning model 110 processes the feature maps 108a, 108b, and possibly the MSI images 102 and OCT image 104, and produces one or more estimated biomarker segmentation maps. For example, the output of the machine learning model 110 may be a three-dimensional array in which each two-dimensional array along a third dimension is an estimated biometric segmentation map corresponding to a pathology. Although two feature maps 108a, 108b are shown, the machine learning model 110 may process any number of feature maps, and possibly any number of images used to generate the feature maps, in a like manner for any number of imaging modalities.

A training algorithm 204 compares the one or more estimated biomarker segmentation maps to the one or more biomarker segmentation maps 118 of the training data entry 200. The training algorithm 204 then updates one or more parameters of the machine learning model 110 according to differences between each estimated biomarker segmentation map for a pathology and the corresponding biomarker segmentation map 118 for that pathology.

As described above with respect to FIG. 1, the machine learning models 114, 116 takes as inputs the feature map 112 of the machine learning models 110. The machine learning models 114, 116 may be trained after the machine learning model 110 is trained with some or all of the training data entries 200.

For each training data entry 200, the machine learning models 114, 116 receive the feature maps 112 obtained from processing the MSI images 102 and OCT image 104 of the training data entry 200 with the machine learning models 106a, 106b, 110. As noted above, the feature map 112 may be the outputs of a hidden layer of the machine learning models 110, i.e., a layer other than the final layer that outputs the one or more estimated biomarker segmentation maps. The machine learning models 114, 116 may also take as inputs the MSI images 102 and OCT image 104, though in other embodiments, only the feature map 112 is used.

The machine learning model 114 processes the feature map 112, and possibly images 102, 104 from the training data entry 200, and produces one or more estimated biomarker segmentation maps. Where three or more imaging modalities are used, images according to the three or more imaging modalities from the training data entry 200 may be processed by the machine learning model 114 along with the feature map 112 obtained from the images. The output of the machine learning model 114 may be a three-dimensional array in which each two-dimensional array along a third dimension is an estimated biometric segmentation map corresponding to a pathology.

A training algorithm 206a compares the one or more estimated biomarker segmentation maps to the one or more biomarker segmentation maps 118 of the training data entry 200. The training algorithm 206a then updates one or more parameters of the machine learning model 114 according to differences between each estimated biomarker segmentation map for a pathology and the corresponding biomarker segmentation map 118 for that pathology.

The machine learning model 116 processes the feature map 112, and possibly the MSI images 102 and OCT image 104, and produces one or more estimated diagnoses and an estimated severity score for each estimated diagnosis. Where three or more imaging modalities are used, images according to the three or more imaging modalities from the training data entry 200 may be processed by the machine learning model 116 along with the feature map 112 obtained for the images.

The output of the machine learning model 116 may be a vector, in which each element of the vector, if nonzero, indicates a pathology is estimated to be present. The output of the machine learning model 116 may also be text enumerating one or more dominant pathologies estimated to be present. The output of the machine learning model 116 may further include a severity score for each pathology estimated to be present, such as a vector in which each element corresponds to a pathology and a value for an element indicates the severity of the corresponding pathology.

A training algorithm 206b compares the estimated diagnoses and corresponding severity scores to the disease diagnoses 120 and severity score 122 of the training data entry 200. The training algorithm 206b then updates one or more parameters of the machine learning model 116 according to differences between the estimated diagnoses and corresponding severity scores and the disease diagnoses 120 and severity score 122 of the training data entry 200.

Referring to FIG. 2B, in some embodiments, training of one or both of the machine learning models 106a, 106b may be performed by an unsupervised training algorithm 210a, 210b respectively. FIG. 2B shows training with images 102, 104 with the understanding that one or more machine learning models for additional or alternative imaging modalities can be trained in the same manner.

For the embodiment of FIG. 2B, the machine learning models 110, 114, 116 may be as described above with respect to FIG. 2A. In some embodiments, only one of the machine learning models 106a, 106b is trained using an unsupervised training algorithm 210a, 210b whereas the other is rained using a supervised training algorithm 202 as described above with respect to FIG. 2A. For the unsupervised machine learning algorithms 210a, 210b, labeled training data entries are not used.

The machine learning model 106a may be trained using a corpus of sets of MSI images 102. The corpus may be curated to include a large number of sets of MSI images, e.g., retinal images, of healthy eyes without pathologies present and a small fraction, e.g., less than 5 percent or less than 1 percent of the corpus, corresponding to one or more pathologies. The sets of MSI images 102 may or may not be labeled as to whether the set of images 102 represent a pathology and/or the specific pathology represented.

The unsupervised training algorithm 210a processes the corpus using the machine learning model 106a and trains the machine learning model 106a to identify and classify anomalies detected in the sets of MSI images 102 of the corpus. The unsupervised training algorithm 210a may be implemented using any approach for performing anomaly detection or other unsupervised machine learning known in the art. The output of the machine learning model 106a may be an image having the same dimensions as an individual MSI image 102 with pixels representing anomalies being labeled.

The machine learning model 106b may be trained using a corpus of OCT images 104. The corpus may be curated to include a large number of OCT images, e.g., retinal images, of healthy eyes without pathologies present and a small fraction, e.g., less than 5 percent or less than 1 percent of the corpus, corresponding to one or more pathologies. The OCT images 104 may or may not be labeled as to whether the set of images 102 represent a pathology and/or the specific pathology represented.

The unsupervised training algorithm 210b processes the corpus using the machine learning model 106b and trains the machine learning model 106a to identify and classify anomalies detected in the OCT images 104 of the corpus. The unsupervised training algorithm 210b may be implemented using any approach for performing anomaly detection or other unsupervised machine learning known in the art. The output of the machine learning model 106b may be an image having the same dimensions as each OCT image 104 with pixels representing anomalies being labeled.

The sets of MSI images 102 and OCT images 104 used to train the machine learning models by unsupervised training algorithms 210a, 210b may include images 102, 104 from the training data entries 200 used to train the other machine learning models 110, 114, 116. The sets of MSI images 102 and OCT images 104 may further be augmented with images of healthy eyes to facilitate the identification of anomalies corresponding to pathologies. The sets of MSI images 102 and OCT images 104 may be constrained to be the same size and may be aligned with one another. For example, although images 102, 104 are of a plurality of different eyes, the images 102, 104 may be aligned to place a representation of a center of the fovea of the retina at substantially the center of each image 102, 104, e.g., within 1, 2, or 3 pixels. Some other feature may be used for alignment, such as the fundus. Although images 102, 104 are of a plurality of different eyes, the images 102, 104 may also be scaled such that anatomy represented in the images is substantially the same size. For example, images 102, 104 may be scaled such that the fovea, fundus, or one or more other anatomical features are the same size.

Once trained, the machine learning models 106a, 106b may provide outputs to the machine learning model 110 (see FIGS. 1 and 2A) in the form of one or both of feature maps 108a, 108b that are the outputs of one or more hidden layers of the machine learning models 106a, 106b, respectively. Alternatively, the final outputs of the machine learning models 106a, 106b, e.g., images with anomaly labels, may be used as the inputs to the machine learning model 110.

Referring to FIG. 2C, in a refinement to the unsupervised machine learning approach of FIG. 2B, a supervised training algorithm 212b may compare the output of the machine learning model 106a to the output of the machine learning model 106b for a given set of MSI images 102 and an OCT image 104 of the same patient eye captured substantially simultaneously as defined above. The supervised training algorithm 212b may then adjust parameters of the machine learning model 106b according to the comparison in order to train the machine learning model 106b to identify the same anomalies detected by the machine learning model 106a. Note that the opposite approach may alternatively or additionally be used: the output of the machine learning model 106b may be used by a supervised training algorithm 212b, or a different supervised training algorithm 212a, to train the machine learning model 106a to identify anomalies identified by the machine learning model 106b.

In some implementations, training may proceed in various phases, each phase using one of the training approaches described above with respect to FIGS. 2A, 2B, and 2C. In a first example, machine learning models 106a, 106b are first trained using the supervised machine learning approach of FIG. 2A; the machine learning models 106a, 106b may then be trained using the unsupervised approach of FIG. 2B; and then the machine learning model 106b is further trained based on the output of the machine learning model 106a (and/or vice versa) according to the approach of FIG. 2C. In a second example, only unsupervised learning is used: the machine learning models 106a, 106b are individually trained using the unsupervised approach of FIG. 2B followed by further training machine learning model 106b based on the output of the machine learning model 106a and/or training the machine learning model 106a based on the output of the machine learning model 106b according to the approach of FIG. 2C.

FIG. 2C shows training with images 102, 104 with the understanding that one or more machine learning models for additional or alternative imaging modalities can be trained in the same manner. In particular, the output of a machine learning model according to one imaging modality may be used to train one or more other machine learning models according to one or more other imaging modalities in the same manner. The outputs of two or more first machine learning models for one or more first imaging modalities may be concatenated or otherwise combined and used to train one or more second machine learning models for one or more second machine learning models using the approach of FIG. 2C.

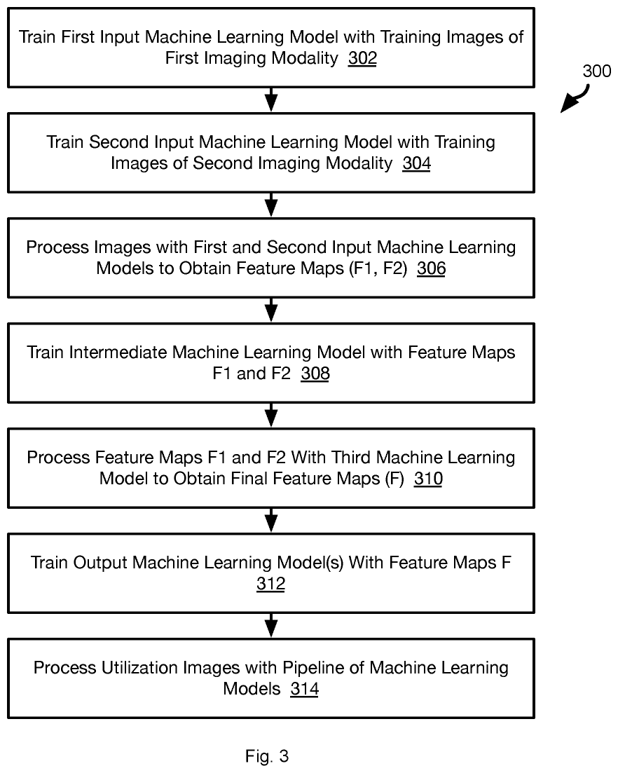

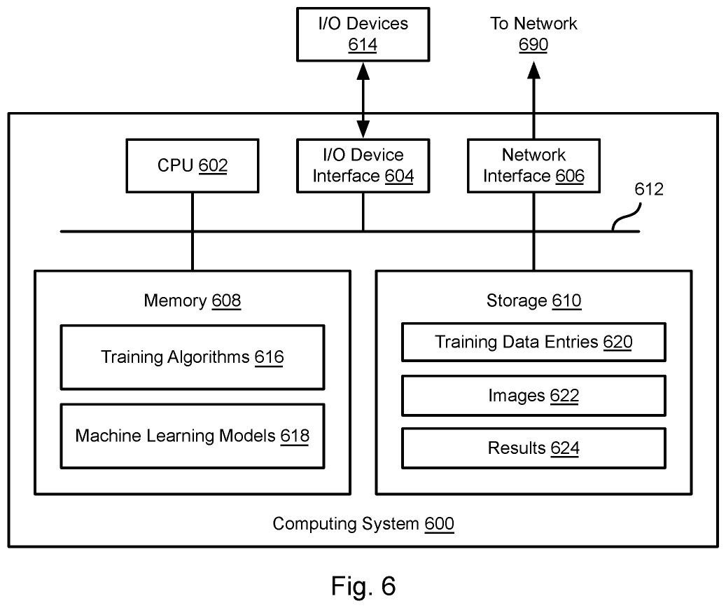

Referring to FIG. 3, the illustrated method 300 may be executed by a computer system, such as the computing system 600 of FIG. 6. The method 300 includes training, at step 302, a first input machine learning model with training images of a first imaging modality. For example, step 302 may include the machine learning model 106a with MCI images 102 according to any of the approaches described above with respect to FIGS. 2A to 2C.

The method 300 includes training, at step 304, a second input machine learning model with training images of a second imaging modality. For example, step 304 may include the machine learning model 106b with OCT images 104 according to any of the approaches described above with respect to FIGS. 2A to 2C.

The method 300 includes processing, at step 306, images according to the first imaging modality with the first input machine learning model to obtain input feature maps F1 and processing images according to the second imaging modality with the second input machine learning model to obtain input feature maps F2. The feature maps F1 and F2 may be outputs of hidden layers of the first and second input machine learning models, respectively. Step 304 may include processing MSI images 102 using the machine learning model 106a and processing OCT images 104 using the machine learning model 106b to obtain feature maps 108a, 108b as described above with respect to FIGS. 1 and 2A. As noted above, MSI images 102 and OCT images 104 may be part of a common training data entry 200 such that the MSI images 102 and OCT images 104 are of the same patient eye and captured substantially simultaneously.

The method 300 includes training, at step 308, an intermediate machine learning model with feature maps F1 and F2. Specifically, a plurality of pairs of feature maps F1 and F2 may each be processed by the intermediate machine learning model and the output of the intermediate machine learning model may be used to train the intermediate machine learning model. Each pair of feature maps F1 and F2 may be obtained for images of the first and second modality that are images of the same patient eye and captured substantially simultaneously. Step 308 may include processing the images used to obtain each pair of feature maps F1 and F2 using the intermediate machine learning model. Step 308 may include training a machine learning model 110 using feature maps 108a, 108b and training data entries 200 as described above with respect to FIG. 2A.

The method 300 includes processing, at step 310, feature pairs of feature maps F1 and F2, and possibly the training images used to obtain the feature maps F1 and F2 of each pair, with the intermediate machine learning model to obtain final feature maps F. The final feature maps F may be obtained from the output of a hidden layer of the intermediate machine learning model. Step 310 may include processing feature maps 108a, 108b, and possibly corresponding images 102, 104, using the machine learning model 110 to obtain feature maps 112 as described above with respect to FIGS. 1 and 2A.

The method 300 includes training, at step 312, one or more output machine learning models with the feature maps F. The one or more output machine learning models may be trained to output, for a given feature map F, an estimated representation of a pathology represented in the training images used to generate the feature map F using the first and second input machine learning models and the intermediate machine learning model. The one or more output machine learning models may take as an input the images according to the first and second imaging modalities that were used to generate the feature map F. Step 312 may include training one or both of machine learning models 114, 116 using the feature map 112 and possibly corresponding images 102, 104, to output some or all of a biomarker segmentation map 118, disease diagnosis 120, and a severity score 122.

The method 300 may include processing, at step 314, utilization images according to the first and second imaging modalities according to a pipeline of the first and second input machine learning models, the intermediate machine learning model, and the one or more output machine learning models. Specifically, one or more of the utilization images according to the first imaging modality are processed using the first input machine learning model to obtain a feature map F1; one or more of the utilization images according to the second imaging modality are processed using the second input machine learning model to obtain a feature map F2; the feature maps F1 and F2, and possibly the utilization images, are processed using the intermediate machine learning model to obtain a feature map F; and the feature map F, and possibly the utilization images, are processed by the one or more output machine learning models to obtain an estimated representation of a pathology represented in the utilization images. The estimated representation may be output to a display device or stored in a storage device for later usage or subsequent processing. Feature maps (F1, F2, F) may additionally be displayed or stored

For example, step 314 may include processing utilization images 102, 104, i.e., images 102, 104 that are not part of a training data entry 200, using the machine learning models 106a, 106b, respectively, to obtain feature maps 108a, 108b, respectively, as described above with respect to FIG. 1. The feature maps 108a, 108b, and possibly the utilization images 102, 104, may be processed using the machine learning models 110 to obtain a feature map 112. The feature map 112, and possibly the utilization images 102, 104, may be processed by one or both of the machine learning models 114, 116 to obtain a biomarker segmentation map 118, disease diagnosis 120, and severity score 122.

The steps 302-314 may be performed in order, i.e. the first and second input machine learning models are trained, followed by training the intermediate machine learning model, followed by training the one or more output machine learning models, followed by utilization. Steps 302-314 may additionally or alternatively be interleaved, i.e., the first and second input machine learning models, the intermediate machine learning model, and the one or more output machine learning models being trained as a group. For example, in a first stage, the first and second input machine learning models, the intermediate machine learning model, and the one or more output machine learning models are trained separately in the order listed and, in a second stage, training continues as a group, i.e., subsequent to an iteration including processing a set of images according to the pipeline, some or all of the first and second input machine learning models, the intermediate machine learning model, and the one or more output machine learning models may be updated as part of the iteration by a training algorithm according to the outputs of the first and second input machine learning models, the intermediate machine learning model, and the one or more output machine learning models, respectively. Training individually or as a group may continue during the utilization step 314, particularly unsupervised learning as described with respect to FIGS. 2B and/or 2C.

Step 314 may be performed by a different computer system than is used to perform steps 302-312. For example, the pipeline including the first, second, third, and one or more output machine learning models may be installed on one or more other computer systems for use by surgeons or other health professionals.

Although the method 300 is described with respect to two imaging modalities, three or more imaging modalities may be used in a like manner. For example, suppose there are imaging modalities IMᵢ, i=1 to N, where N is greater than or equal to two. For a given training data entry, requirements of substantially identical scaling, alignment, and simultaneous imaging of the same eye of a patient may be met by images of the imaging modalities IMᵢ. There may be input machine learning models MLᵢ, i=1 to N, each machine learning model MLᵢ corresponding to an imaging modality IMᵢ and each generating a corresponding feature map Fᵢ by processing one or more images of the corresponding imaging modality IMᵢ. Each input machine learning model MLᵢ may be trained with images of the corresponding imaging modality according to any of the approaches described above for training the machine learning models 106a, 106b.

The intermediate machine learning model in such embodiments would therefore take N feature maps Fᵢ, i=1 to N, as inputs, and possibly the training images used to generate the feature maps Fᵢ. The output machine learning model would take as inputs the final feature map F and possibly the training images used to generate the feature maps Fᵢ. The intermediate machine learning model and output machine learning model are trained as described above with respect to the machine learning model 110 and the machine learning models 114, 116.

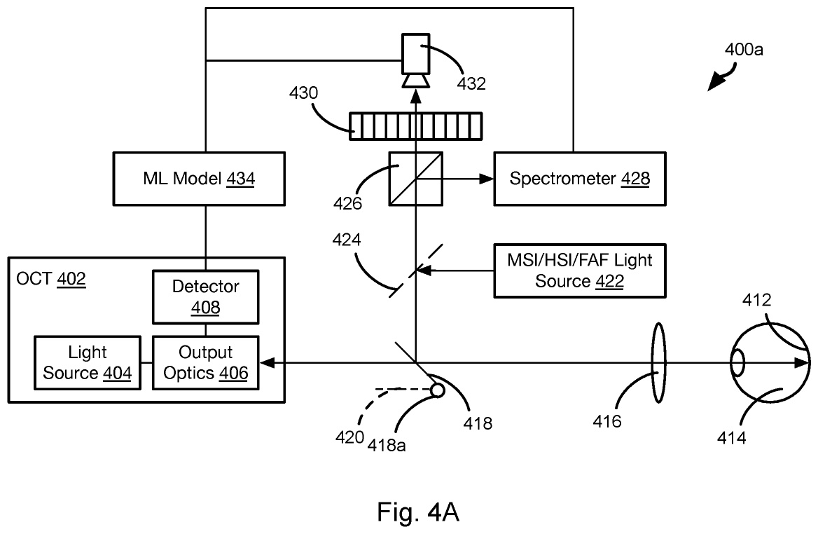

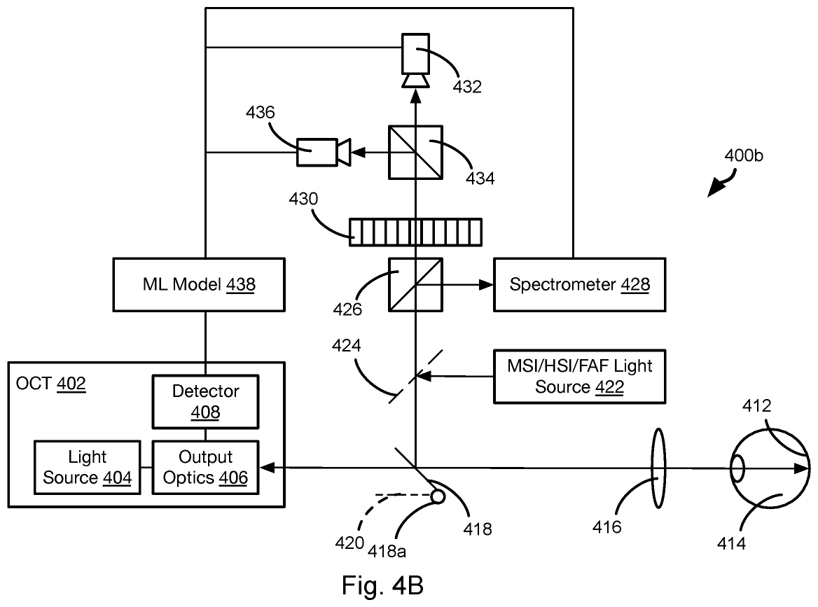

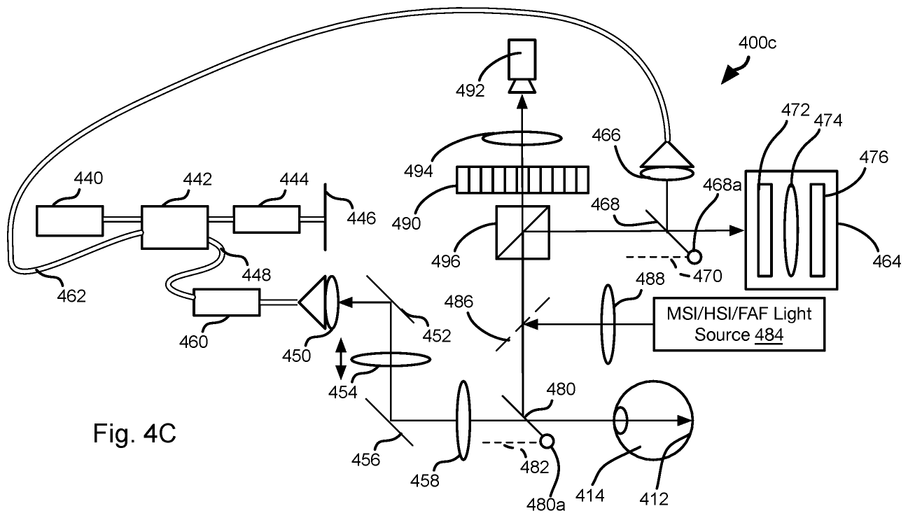

FIGS. 4A, 4B, and 4C illustrate example systems 400a, 400b, 400c that may be used to capture images of multiple imaging modalities substantially simultaneously and detect some or virtually all known retinal pathologies. For example, diabetic retinopathy is becoming increasingly common. Age related macular degeneration (AMD) and glaucoma are also relatively common among the elderly. The elderly will typically experience at least some loss of vision due to one or more retinal pathologies. Since many retinal diseases are progressive, early detection is critical to improve life quality and reduce blindness.

Generally, ophthalmic clinic settings have various tools, including separate instruments, such as OCT, fundus camera, scanning laser ophthalmoscope (SLO), etc. At the early stages of retinal disease, it is difficulty in differentiate between diseases. Images of multiple imaging modalities are helpful for disease differentiation.

OCT images provide high-resolution structural information on all layers of the retina on a micron scale. However, structural changes are not likely in early stage of a disease. Metabolic status will typically have been abnormal for time before any structural change is detectable with an OCT.

A color fundus camera provides color fundus images but does not cover fluorescence range of 500 nm to 600 nm, which is important for detecting fluorophores deposited in the retinal pigment epithelium (RPE), which are present in the early stage of AMD and could develop into drusen or atrophy.

Fundus autofluorescence (FAF) enables imaging of indicators of retinal degeneration and focal hypo- and hyperpigmentation at the level of the RPE. FAF is the most reliable imaging modality to detect, delineate, quantify, and monitor progression of outer retinal atrophy. BlinD (basal linear deposit) and BLamD (basal laminar deposit) in RPE are precursors of AMD and can be visualized using FAF but not an OCT.

Currently there is no single tool that can reliably perform early-stage detection, risk assessment, and progression monitoring for retinal diseases. FIGS. 4A, 4B, and 4C illustrate example systems 400a, 400b, 400c that are able to provide this functionality. Images obtained using the systems 400a, 400b, 400c may be processed using a machine learning model to further facilitate early-stage detection, risk assessment, and monitoring of progression of retinal diseases.

Referring specifically to FIG. 4A, a system 400a includes an OCT 402. The OCT 402 may be implemented as any OCT known in the art, which may include a light source 404, output optics 406, and a detector 408. The light source 404 may be a coherent light source, such as laser or laser diode. The light source 404 may also be a low-coherence broadband light source. The output optics 406 may include a scanning mirror, focusing optics, and a mechanism for translating a depth of focus of the OCT 402, such as for a time-domain OCT. The detector 408 may include a spectrometer, such as a diffraction grating and a charge coupled device (CCD), complimentary metal oxide semiconductor (CMOS) sensor, or other detector. The output of the detector 408 is either an image or a stream of samples that may be organized into an image based on the state of the OCT 402 when each sample was detected.

The light from the light source 404 is directed by the output optics 406 onto the retina 412 of an eye 414 of a patient. The light from the light source 404 may pass through one or more lenses 416 in order to focus the light on the retina 412. The light reflected from the retina 412 arrives back at the output optics 406 and at least a portion thereof is directed to the detector 408.

An actuated mirror 418, such as a toggle switch mirror, may be positionable as illustrated or actuated to the position 420. When the OCT 402 is in use, the actuated mirror 418 is placed in the position 420 and light reflected from the retina 412 is allowed to reach the OCT 402 without interaction with the mirror 418. The mirror 418 may be positioned as shown in FIG. 4A in order to capture images according to one or more other imaging modalities. For example, a reflective surface of the mirror 418 may be oriented at an approximately 45 degree (e.g., +/−2 degrees) angle relative to an optical axis of the lens 416 and/or the eye 414. The mirror 418 may be manually switched between the illustrated positions or may be coupled to an electrical actuator 418a.

A light source 422 may be used to illuminate the retina 412 when imaging according to the one or more other imaging modalities. The one or more other imaging modalities may include some or all if MSI, HSI, fundus auto fluorescence (FAF), FAF spectrum, infrared, ultraviolet, or other imaging modality. The light source 422 may include (a) a single light source suitable for the one or more other imaging modalities, (b) a single light source that may be operated in different ways (intensity and/or spectrum) for different imaging modalities, or (c) multiple light sources, each light source being used for a different imaging modality. The light source 422 may be implemented as a broadband light source embodied as one or more LEDs.

Light from the light source 422 may be incident on a mirror 424, such as an annular aperture mirror, beam splitter, or other mirror capable of partial reflection and transmission. The mirror 424 directs at least a portion of the light from the light source 422 onto the mirror 418, which directs the light onto the retina 412, such as by way of the lens 416.

Light reflected from the retina 412 is directed by the mirror 418 back to the mirror 424, which permits at least a portion of the reflected light to pass therethrough. At least a portion of the reflected light may be directed into a spectrometer 428. The spectrometer 428 captures spectral information for the reflected light.

A portion of the light reflected from the retina 412 may additionally or alternatively be reflected through a filter 430 onto a camera 432. The camera 432 may be a monochrome or color camera. The filter 430 may be a filter wheel including a plurality of filters, each filter corresponding to a different band of wavelengths. A plurality of images of the reflected light may be captured, each image being captured with a different filter of the plurality of filters interposed between the camera 432 and the retina 412. The plurality of images may therefore constitute an MSI or HSI image. The filter 430 may include an electronically controlled actuator for selecting among the plurality of filters or may be manually adjustable.

Where both a spectrometer 428 and camera 432 are used, a beam splitter 426 may be positioned such that light reflected from the retina 412 is both (a) transmitted through the beam splitter 426 onto one of the camera 432 and the spectrometer 428 and (b) reflected from the beam splitter 426 onto the other of the spectrometer 428 and the camera 432.

An OCT image output by the detector 408, an FAF image and/or FAF spectral information obtained using spectrometer 428, and MSI or HSI images obtained using the camera 432 may be input to a machine learning model 438. The machine learning model 438 is trained to some or all of (a) identify anatomy and features corresponding to pathologies of the retina, (b) diagnose one or more pathologies of the retina, and (c) estimate a severity for one or more pathologies.

The machine learning model 438 may be embodied as the system 100 as described above that has been trained according to any of the embodiments described above. Where three or more imaging modalities are implemented using the system 400a, the system 100 may be configured as described above to include three or more machine learning models 106a, 106b generating three or more feature maps 108a, 108b that are input to the intermediate machine learning model 110. Where three or more imaging modalities are implemented using the system 400a, training data entries 200 used to train the machine learning models 106a, 106b, 110, 114, 116 may include images for the three or more imaging modalities in addition to or in place of the MSI images 102 and OCT image 104 described above. The machine learning models 106a, 106b, 110, 114, 116 may be trained in the same manner as described above, with each machine learning model 106a, 106b being trained with images of an imaging modality corresponding to that machine learning model 106a, 106b.

The system 400a has many advantages when used in combination with the system 100. The system 400a can readily capture images of a plurality of imaging modalities substantially simultaneously without requiring the patient to move to another device. The system 400a may likewise be configured such that the plurality of images according to the plurality of imaging modalities are substantially identically scaled (e.g., within 0.01 percent along each dimension) and substantially aligned, e.g., within 0.1 mm, 0.01 mm, or within 1 μm measured with respect to features of the retina represented in the plurality of images. Substantially identical scaling may be obtained by calibrating the magnification of each imaging modality. Substantially alignment may be obtained by substantially aligning the optical axis of each imaging modality with the center of images obtained using each imaging modality.

Images of the plurality of imaging modalities obtained using the system 400a may be processed by the machine learning model 438 upon capture. With readily available computing capacity, the results of the processing can be available within minutes. An ophthalmologist, surgeon, or other health professional can therefore immediately provide a diagnosis to a patient with regards to virtually any retinal disease.

Referring to FIG. 4B, the system 400b may be modified relative to the system 400a by the use of an additional camera 436. Light reflected from the retina 412 may be directed to both cameras 432, 436 by means of a beam splitter 434. The cameras 432, 436 may capture different types of images. For example, the cameras 432, 436 may capture two or more of the following types of images: color (RGB), infrared, ultraviolet, MSI, HSI, and FAF. Images from the camera 436 may be processed using the machine learning model 438 along with other images according to other imaging modalities provided by the system 400b as describe above with respect to FIG. 4A.

Referring to FIG. 4C, a spectral domain OCT (SD-OCT) may be modified to achieve the illustrated system 400c in order to capture images according to a plurality of other imaging modalities (MSI, HSI, FAF, color, infrared, ultraviolet).

The SD-OCT includes a light source 440. The light source 440 may be a low-coherence light source such as a broadband light source generating light across the entire visible spectrum (e.g., 380 to 700 nm) and possibly also in the infrared and/or ultraviolet spectrum. The light source 440 may be embodied as one or more light emitting diodes (LED).

Light from the light source 440 is input to a fiber optic coupler 442. A portion of the light is transmitted into a dispersion compensator 444. The light passes through the dispersion compensator 444, is incident on a mirror 446, and is reflected back through the dispersion compensator 444 to the fiber optic coupler 442.

Light from the light source 440 is also coupled by the fiber optic coupler 442 to an optical fiber 448. The optical fiber 448 conducts light to a lens 450 that directs the light received from the optical fiber 448 onto a scanning mirror 452. The scanning mirror 452 may be a single mirror that is actuated along two orthogonal rotational axes in order to scan light across a two-dimensional region of the retina 412. Alternatively, the scanning mirror 452 may be embodied as two mirrors, each being rotated about one of two orthogonal rotational axes. For example, the scanning mirror 452 may be embodied as a galvo mirror and a resonant scanner.

Light reflected from the scanning mirror 452 passes through a scanning lens 454. The scanning lens 454 is actuated along the optical axis of the scanning lens 454 in order to change a depth of focus of light form the light source 440 that reaches the retina 412. The scanning lens 454 is therefore translated to different positions to image different layers of the retina 412. The scanning lens 454 may be mechanically actuated or may vary the depth of focus by electronically, such as by implementing the scanning lens 454 as an optofluidic lens.

The light emitted by the scanning lens 454 may be directed by one or more other components onto the retina 412. For example, one or more mirrors 456 may change the direction of the light emitted from the lens 454 and one or more lenses 458 may focus the light emitted from the lens 454 onto the retina 412. The position and/or orientation of the one or more mirrors 456 and one or more lenses 458 may be tunable.

In some embodiments, adaptive optics (AO) 460 may be positioned in the optical path between the lens 450 and the fiber optic coupler 442. The adaptive optics 460 improve the quality of images obtained using an SD-OCT and the properties of the AO 460 may be selected according to any approach known in the art of SD-OCT design.

Light reflected from the retina 412 follows the reverse of the path traversed by light traveling from the fiber optic coupler 442 to the retina 412. Upon arriving at the fiber optic coupler 442, at least a portion of the light reflected from the retina 412 along with at least a portion of the light returning from the dispersion compensator 444 are coupled to optical fiber 462. Optical fiber 462 directs light onto a spectrometer 464, such as by way of an output lens 466 that receives light output by the optical fiber 462 and focuses or collimates the light that is input to the spectrometer 464.

In some embodiments, the fiber optical coupler 442 is coupled to the light source 440 and dispersion compensator by multi-mode optical fibers and the optical fibers 462, 448 are single-mode optical fibers

The spectrometer 464 may additionally be used for one or more other imaging modalities. Accordingly, a mirror 468, beam splitter, or other optical element may be used to enable light from multiple sources to be directed into the spectrometer 464. In the illustrated embodiment, the mirror 468 is an actuated mirror. The mirror 468 may be placed in the orientation shown to reflect light from the output lens 466 into the spectrometer 464. The mirror 468 may be moved to the orientation 470 to permit light from one or more other sources to enter the spectrometer 464. The mirror 468 may be manually switched between the illustrated positions or may be coupled to an electrical actuator 468a.

The spectrometer 464 may be implemented using any type of spectrometer known in the art. In the illustrated embodiment, the spectrometer 464 includes a diffraction grating 472, a lens 474, and a detector 476, such as a CCD or CMOS sensor. Light incident on the grating 472 forms a wavelength dependent fringe pattern that is focused by the lens 474 onto the detector 476. Accordingly, the light incident on each point on the detector 476 can be mapped to a particular wavelength. The output of the detector 476 may therefore be processed to measure the spectrum of light entering the spectrometer 464. Inasmuch as light from the light source 440 is scanned across the retina 412, the reflectivity spectrum of a spot on the retina 412 may be obtained from each spectrum measurement of the spectrometer 464.

A mirror, beam splitter, or other optical element may be used to direct light for one or more other imaging modalities onto the retina 412. For example, an actuated mirror 480, such as a toggle switch mirror, may be positionable as illustrated or actuated to the position 482. When SD-OCT imaging is performed, the actuated mirror 480 is placed in the position 482 and light is allowed to travel between the light source 440 and the retina 412 without interaction with the mirror 480. The mirror 480 may be positioned as shown in FIG. 4C in order to capture images according to one or more other imaging modalities. For example, a reflective surface of the mirror 480 may be oriented at an approximately 45 degree (e.g., +/−2 degrees) angle relative to an optical axis of the lens 458 and/or eye 414. The mirror 480 may be manually switched between the illustrated positions or may be coupled to an electrical actuator 480a

A light source 484 may be used to illuminate the retina 412 when imaging according to the one or more other imaging modalities. The one or more other imaging modalities may include some or all if MSI, HSI, FAF, color, infrared, ultraviolet, or other imaging modality. The light source 484 may include (a) a single light source suitable for the one or more other imaging modalities, (b) a single light source that may be operated in different ways (intensity and/or spectrum) for different imaging modalities, or (c) multiple light sources, each light source being used for a different imaging modality. For example, the light source 484 may be a broadband light source implemented using one or more LEDs.

Light from the light source 484 may be incident on a mirror 486, such as an annular aperture mirror, beam splitter, or other mirror capable of partial reflection and transmission. One or more lenses 488 may be interposed between the light source 484 and the mirror 486 to focus light from the light source 484 onto the retina 412 or to collimate light from the light source 484. The mirror 486 directs at least a portion of the light from the light source 484 onto the mirror 480, which directs the light onto the retina 412.

Light reflected from the retina 412 is directed by the mirror 480 back to the mirror 486, which permits at least a portion of the reflected light to pass therethrough. A portion of the reflected light may be directed into the spectrometer 464.

A portion of the light reflected from the retina 412 may additionally or alternatively be reflected through a filter 490 onto a camera 492. In some embodiments, one or more lenses 494 may be interposed between the filter 490 and the camera 492. The camera 492 may be a monochrome, color, infrared, ultraviolet, or other type of camera. The filter 490 may be a filter wheel including a plurality of filters, each filter corresponding to a different band of wavelengths. The filter wheel may be manually adjustable or include an electronically controlled actuator. A plurality of images of the reflected light may be captured, each image being captured with a different filter of the plurality of filters interposed between the camera 492 and the retina 412. The plurality of images may therefore constitute an MSI or HSI image. The system 400c may be modified similarly to the system 400b to use multiple cameras to image light passing through the filter 490.

Where both the spectrometer 464 and the camera 492 are used to image light from the light source 484, a beam splitter 496 may be positioned such that light reflected from the retina 412 is both (a) transmitted through the beam splitter 496 onto one of the camera 492 and the spectrometer 464 and (b) reflected from the beam splitter 496 onto the other of the spectrometer 464 and the camera 492.

The output of the spectrometer 464 when the system 400c is operating as an SD-OCT is used to form an OCT image as known in the art of SD-OCT. When mirror 480 is used to direct light from the light source 484 onto the retina 412, the output of the spectrometer 464 may be used to form an FAF image and outputs of the camera 492 may be used to form MSI or HSI images. Any other cameras used may produce FAF, color, infrared, ultraviolet, or other types of images. The OCT, MSI or HSI, FAF, and any other images obtained using the system 400c may be processed using the machine learning model 438 as described above.

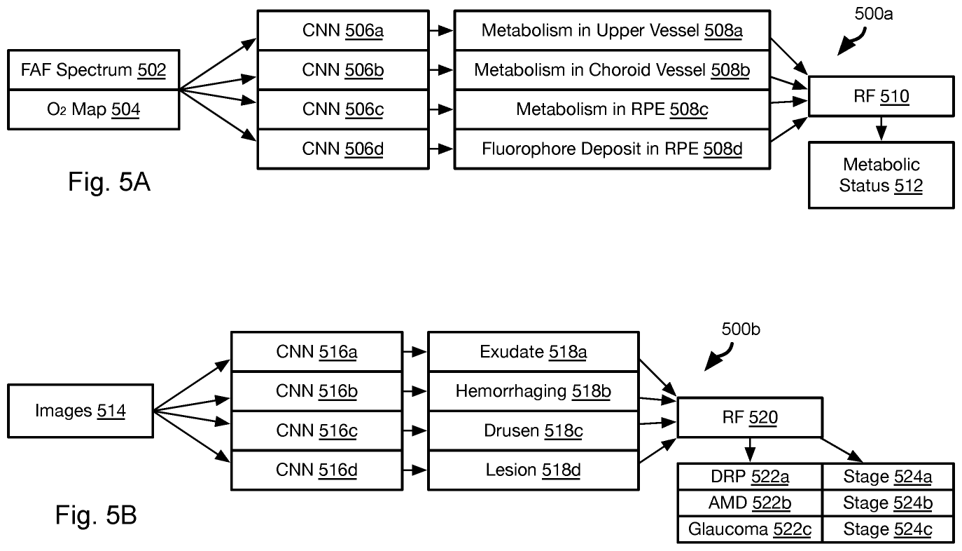

Nom FIGS. 5A and 5B illustrate additional machine learning models 500a, 500b that may be used in addition to the system 100 or in place of the system 100 as the machine learning model 438.

Referring specifically to FIG. 5A, the machine learning model 500a may take as an input one or both of an FAF spectrum 502 and an oxygen map 504. An oxygen map measures the level of oxygen saturation within blood vessels of the retina 412. The oxygen map is obtained by analyzing the spectrum reflected from points within the retina 412. Accordingly, the oxygen map may be obtained using the output of the spectrometer 428, 464 using any approach known in the art for generating an oxygen map. The FAF spectrum 502 may likewise be obtained from the spectrometer 428, 464.

The FAF spectrum 502 and oxygen map 504 may each be input to a plurality of machine learning models 506a-506d that each produce a corresponding output 508a-508d that describes a particular metric, item of anatomy, or feature corresponding to a pathology. In the illustrated embodiment, the machine learning models 506a-506d are convolution neural networks (CNN) 506a-506d. However, the machine learning models 506a-506d may also each be embodied as a deep neural network (DNN), a recurrent neural network (RNN), a region-based CNN (R-CNN), an autoencoder (AE), or other type of machine learning model.

The outputs 508a-508d may include, for example, the metabolism in the upper vessels of the retina (e.g., anywhere between the Bruch's membrane and the vitreous), metabolism in choroid vessels, the metabolism in the RPE, fluorophore deposits in the RPE, or other items of anatomy or features corresponding to a pathology that may be represented in spectral information included in the FAF spectrum 502 and/or oxygen map 504.

The outputs 508a-508d may be input to an output machine learning model 510. In the illustrated embodiment, the output machine learning model 510 is a random forest. However, other machine learning models may be used such as a long short term memory (LSTM) machine learning model, a generative adversarial network (GAN) machine learning model, logistic regression machine learning model, or other type of machine learning model.

The output machine learning model 510 outputs a metabolic status 512 for the retina 412 represented by the FAF spectrum and the oxygen map 504. The metabolic status 512 may reflect such information as blood flow, oxygen saturation, clearing of waste products, or other information. The metabolic status 512 may be in the form of a numerical values indicating an overall metabolic status of the retina 412, e.g., low indicating poor health and high indicating good health. The metabolic status 512 may be a plurality of numerical values each corresponding to one aspect of the metabolism of the retina 412. The metabolic status may be stored and/or output to a display device. For example, a representation of some or all of the FAF spectrum 502, oxygen map 504, outputs 508a-508d, and metabolic status may be stored and/or output to a display device for evaluation by an ophthalmologist, surgeon, or other health professional.

The machine learning models 506a-506d and machine learning model 510 may be trained using training data entries. For example, each training data entry may include an FAF spectrum 502 and oxygen map 504 for a retina 412 as an input and, as desired outputs, outputs 508a-508d as determined by a human labeler and a metabolic status 512 as determined by a human labeler.

Accordingly, a training algorithm may process the FAF spectrum 502 and oxygen map 504 of a training data entry with the machine learning models 506a-506d to obtain estimated outputs 508a-508d that are compared to the outputs 508a-508d of the training data entry. The training algorithm then updates parameters of the machine learning models 506a-506d according to the comparison.

The output machine learning model 510 may be trained by processing the outputs 508a-508d from the training data entry with the machine learning model 510 to obtain an estimated metabolic status 512. The training algorithm then compares the estimated metabolic status 512 to the metabolic status 512 from the training data entry and updates the output machine learning model 510 according to the comparison.

Referring specifically to FIG. 5B, the machine learning model 500b takes one or more images 514 of a retina 412 as inputs. The one or more images 514 may include an OCT image, MSI image, HSI image, FAF image, monochrome or color images, infrared images, ultraviolet images, or other images of the retina 412.

The one or more images 514 may be input to a plurality of machine learning models 516-516d that each produce a corresponding output 518a-518d that describes a particular metric, item of anatomy, or feature corresponding to a pathology. In the illustrated embodiment, the machine learning models 516a-516d are convolution neural networks (CNN) 516a-516d. However, the machine learning models 516a-516d may also each be embodied as a deep neural network (DNN), a recurrent neural network (RNN), a region-based CNN (R-CNN), an autoencoder (AE), or other type of machine learning model.

The outputs 518a-518d may include, for example, representations of exudate, hemorrhaging, drusen, lesions, or other items of anatomy or features corresponding to a pathology that may be represented in some or all of the one or more images 514.

The outputs 518a-518d may be input to an output machine learning model 520. In the illustrated embodiment, the output machine learning model 520 is a random forest. However, other machine learning models may be used such as a long short term memory (LSTM) machine learning model, a generative adversarial network (GAN) machine learning model, logistic regression machine learning model, or other type of machine learning model.

The output machine learning model 520 outputs, for each pathology of one or more pathologies, a diagnosis 522a-522c. The output machine learning model 520 may additionally output a stage 524a-524c for each pathology of the one more pathologies that indicates a level of progression of the pathology. For example, the pathologies may include diabetic retinopathy (DRP), age-related macular degeneration (AMD), glaucoma, or other pathologies that may be represented in images of the retina 412. The stage 524a-524c for each pathology may be one of a discrete set of values for each pathology, e.g., a number from 1 to 10, or other set of discrete values used by health professionals to represent the progression of a pathology.

A representation of the diagnoses 522a-522c and stages 524a-524c may be stored and/or output to a display device. A representation of some or all of the one or more images 514, outputs 518a-518d, may be stored and/or output to a display device for evaluation by an ophthalmologist, surgeon, or other health professional.

The machine learning models 516a-516d and machine learning model 520 may be trained using training data entries. For example, each training data entry may include one or more images 514 for a retina 412 as an input and, as desired outputs, outputs 518a-108d, diagnoses 522a-522c, and stages 524a-524c for the diagnoses 522a-522c as determined by a human labeler.

A training algorithm may process the one or more images 514 of a training data entry with the machine learning models 516a-516d to obtain estimated outputs 518a-518d that are compared to the outputs 518a-518d of the training data entry. The training algorithm then updates parameters of the machine learning models 516a-516d according to the comparison.

The output machine learning model 520 may be trained by processing the outputs 518a-518d from the training data entry with the output machine learning model 520 to obtain estimated diagnoses 522a-522c and stages 524a-524c. The training algorithm then compares the estimated diagnoses 522a-522c and stages 524a-524c to the diagnoses 522a-522c and stages 524a-524c from the training data entry and updates the output machine learning model 520 according to the comparison.

The systems and methods disclosed herein provide at least the following advantages:
A single device according to FIG. 4A, 4B, or 4C may be used by an operator to obtain images and spectral information sufficient to identify virtually all retinal pathologies in one sitting of a patient.
The associated machine learning models likewise can output labeled images and diagnoses based on these images during the same visit in which the images were captured.
The associated machine learning models combine information from multiple imaging modalities to perform early detection of retina pathologies.
The convenience with which the images and diagnoses can be obtained enables testing to be performed earlier and more frequently which facilitates both earlier detection and more frequent testing in order to enable the observation of changes over time to assess the progression of pathologies.

FIG. 6 illustrates an example computing system 600 that implements, at least partly, one or more functionalities described herein. An imaging device according to any of FIGS. 4A to 4C may include a computing device having some or all of the attributes of the computing system 600. The computing device may be coupled to one or more cameras 432, 436, 492 and receive images therefrom. The computing device further receive the output of the spectrometer 428, 464 and/or the output of the OCT 402, where the OCT 402 includes a detector 408 other than the spectrometer 428. The computing device may control operation of the systems 400a, 400b, 400c to capture images and transition between imaging modalities as described above, including controlling some or all of the OCT 402, any light sources 422, 484, one or more mirror actuator (e.g., actuators 418a, 480a, 468a), scanning mirror 452, one or more actuators of a scanning lens 454, and an actuator of a filter 430, 490 embodied as a filter wheel or other device enabling selection among a plurality of filters.

The computing device integrated into an imaging device according to any of FIGS. 4A to 4C may further execute the machine learning model 438 and/or machine learning models 500a, 500b. Alternatively, a different computing device having some or all of the attributes of the computing system 600 may receive images and spectral information from the imaging device and process the images and spectral information using the machine learning model 438 and/or machine learning models 500a, 500b.

As shown, computing system 600 includes a central processing unit (CPU) 602, one or more I/O device interfaces 604, which may allow for the connection of various I/O devices 614 (e.g., keyboards, displays, mouse devices, pen input, etc.) to computing system 600, network interface 606 through which computing system 600 is connected to network 690, a memory 608, storage 610, and an interconnect 612.

In cases where computing system 600 is an imaging system, the computing system 600 may further include one or more optical components for obtaining ophthalmic imaging of a patient's eye as well as any other components known to one of ordinary skill in the art.

CPU 602 may retrieve and execute programming instructions stored in the memory 608. Similarly, CPU 602 may retrieve and store application data residing in the memory 608. The interconnect 612 transmits programming instructions and application data, among CPU 602, I/O device interface 604, network interface 606, memory 608, and storage 610. CPU 602 is included to be representative of a single CPU, multiple CPUs, a single CPU having multiple processing cores, and the like.

Memory 608 is representative of a volatile memory, such as a random access memory, and/or a nonvolatile memory, such as nonvolatile random access memory, phase change random access memory, or the like. As shown, memory 608 may store training algorithms 616, such as any of the training algorithms 202, 204, 206a, 206b, 210a, 210b, 212a, 212b described herein or training algorithms for training the machine learning models 500a, 500b as described above. The memory 608 may further store machine learning models 618, such as any of the machine learning models 106a, 106b, 110, 114, 116, 438, 500a, 500b described herein.

Storage 610 may be non-volatile memory, such as a disk drive, solid state drive, or a collection of storage devices distributed across multiple storage systems. Storage 610 may optionally store training data entries 620, such as training data entries 200 or training data entries for training the machine learning models 500a, 500b as described above. The storage 610 may store images 622 obtained according to any of the imaging modalities described herein. The storage 610 may store the result of processing images using the machine learning models 618, including possibly storing intermediate results of any of the machine learning models 618.

Note that the computing system 600 used to train the machine learning models 618 may be different from the computing system 600 that utilizes the trained machine learning models 618. Accordingly, the machine learning models 618 and results 624 of the machine learning models 618 may be present without corresponding training algorithms 616 and the training data entries 620.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A system comprising:
a first imaging device configured to capture a first image of a retina of a patient, the first image being an optical coherence tomography (OCT) image; and
a second imaging device configured to capture a second image of the retina of the patient according to an imaging modality other than OCT;
wherein at least one of:
a sensor of the first imaging device is shared with the second imaging device; or
an optical component is configured to select which of the first imaging device and the second imaging device receives light reflected from the retina to the first imaging device or the second imaging device.

2. The system of claim 1, wherein the imaging modality other than OCT is fundus autofluorescence (FAF).

3. The system of claim 2, wherein the sensor of the first imaging device is shared with the second imaging device and the sensor is a spectrometer.

4. The system of claim 3, wherein:
the optical component is a first actuated mirror;
the first imaging device comprises a first light source and the second imaging device comprises a second light source; and
the first actuated mirror is further configured to select which of the first light source and the second light source is used to illuminate the retina.

5. The system of claim 4, further comprising a second actuated mirror, the second actuated mirror configured to select between (a) allowing light from first light source to enter the spectrometer and (b) allowing light from the second light source to enter the spectrometer.

6. The system of claim 1, wherein the imaging modality other than OCT is a multi-spectral imaging camera.

7. The system of claim 1, wherein the first imaging device is a spectral domain OCT (SD-OCT) device.

8. The system of claim 1, further comprising:
one or more processing devices and one or more memory devices coupled to the one or more processing devices, the one or more memory devices storing executable code that, when executed by the one or more processing devices, causes the one or more processing devices to:
receive the first image and the second image; and
process the first image and second image using a machine learning model to obtain at least one of a diagnosis of a retinal pathology and an identification of a feature represented in at least one of the first and second images and corresponding to the retinal pathology.

9. The system of claim 1, further comprising:
one or more processing devices and one or more memory devices coupled to the one or more processing devices, the one or more memory devices storing executable code that, when executed by the one or more processing devices, causes the one or more processing devices to:
receive the first image and the second image;
process the first image and second image using a plurality of first machine learning models to obtain a plurality of outputs representing features of the retina; and
process the plurality of outputs using a second machine learning model to obtain a diagnosis of a retinal pathology.

10. The system of claim 9, wherein the executable code, when executed by the one or more processing devices, further causes the one or more processing devices to process the plurality of outputs using the second machine learning model to obtain a stage of the retinal pathology.

11. A method comprising:
capturing a first image of a retina of a patient with an imaging device, the first image being an OCT image;
reconfiguring the imaging device to an imaging modality other than OCT;
capturing a second image of the retina of the patient with the imaging device with the imaging modality other than OCT; and
processing the first image and the second image using a machine learning model to obtain at least one of a diagnosis of a retinal pathology and a representation of a feature corresponding to the retinal pathology.

12. The method of claim 11, wherein the imaging modality other than OCT is fundus autofluorescence (FAF).

13. The method of claim 12, wherein capturing the first image and capturing the second image is performed using a same sensor.

14. The method of claim 13, wherein the sensor is a spectrometer.

15. The method of claim 14, wherein reconfiguring the imaging device to the imaging modality other than OCT comprises actuating a mirror to transition between (a) allowing light from a first light source that is reflected from the retina to enter the spectrometer and (b) allowing light from a second light source that is reflected from the retina to enter the spectrometer.

16. The method of claim 15, wherein capturing the first image of a retina comprises scanning the retina with the light the first light source.

17. The method of claim 11, wherein the imaging modality other than OCT is multi-spectral imaging.

18. The method of claim 11, wherein capturing the first image of the retina comprises capturing the first image using spectral domain OCT (SD-OCT).

19. The method of claim 11, wherein the machine learning model is an output machine learning model; and
wherein processing the first image and the second image using the machine learning model comprises:
processing the first image and second image using a plurality of input machine learning models to obtain a plurality of outputs representing features of the retina; and
processing the plurality of outputs using the output machine learning model to obtain the diagnosis of the retinal pathology.

20. The method of claim 19, further comprising processing the plurality of outputs using the output machine learning model to obtain a stage of the retinal pathology.
